(19) United States
(12) Patent Application Publication
(10) Pub. No.: US 2025/0319040 A1
(43) Pub. Date: Oct. 16, 2025

(54) DIETARY SLEEP SUPPLEMENT

(71) Applicant: Paspa Labs, LLC, Raleigh, NC (US)

(72) Inventors: Alexander William Paspa, Hickory, NC (US); Maximilian Paul Paspa, Raleigh, NC (US)

(21) Appl. No.: 19/176,577

(22) Filed: Apr. 11, 2025

Related U.S. Application Data
(60) Provisional application No. 63/633,223, filed on Apr. 12, 2024.

(51) Int. Cl. A61K 31/10; A61K 31/4045; A61K 31/405; A61K 31/4415; A61K 33/06; A61K 33/30; A61K 36/754

(52) U.S. Cl. CPC A61K 31/10; A61K 31/4045; A61K 31/405; A61K 31/4415; A61K 33/06; A61K 33/30; A61K 36/754

(57) ABSTRACT

A sleep supplement formulation that improves a person's sleep includes Sulforaphane and at least one additional ingredient selected from the following: Evodia Fruit Extract, Vitamin C, Quercetin, Red Sage, Angelica Dahruica Root, Golden Seal, L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract. A method of mitigating the effects of caffeine on a person's sleep includes orally administering to the person the sleep supplement formulation.

### CROSS-REFERENCE TO RELATED APPLICATIONS

This application claims benefit of and priority to application No. 63/633,223 filed Apr. 12, 2024, the contents of which are expressly incorporated herein by reference in its entirety.

### FIELD OF INVENTION

The present invention relates to a dietary sleep supplement that combats the effects of caffeine prior to sleep to protect deep sleep.

### BACKGROUND

Many people rely on caffeine to boost energy. Working professionals often use caffeine late in the day to stay productive. Athletes and gym goers often use caffeine before a workout for late afternoon or evening productivity boosts.

Ingesting caffeine, particularly later in the day, can negatively affect a person's ability to either fall asleep or enter into deep sleep. A person's sleep cycle begins with a first stage of initially falling asleep during which many body functions slow down and muscles relax. In a second stage of sleep, the body's core temperature drops and eye movements stop, followed by subsequent stages of deep sleep, or slow wave sleep, in which brain waves, heartbeat, and breathing all slow down. The body accomplishes a lot of work during deep sleep, including cell regeneration, strengthening the immune system, increasing blood supply to muscles, promoting growth and repair of tissues and bones, and increasing glucose metabolism in the brain, which supports short-term and long-term memory. Depriving the body of deep sleep can result in negative effects on a person's health, such as a decrease in testosterone, prolonged or limited recovery from injury or illness, and poor insulin regulation.

Caffeine at night has been shown to reduce deep sleep by 20%. If nightly caffeine intake is a habit, this is the equivalent of aging a person by 30 years. The quarter life of caffeine is on average about 12 hours, depending on one's predisposition to metabolizing caffeine. This means that if a person has an energy drink with 300 mg of caffeine at noon, a quarter (75 mg) of that caffeine would still be present in their system at midnight. “Fast caffeine metabolizers” are people who seemingly are not susceptible to the effects of caffeine. However, caffeine has still been shown to negatively affect the deep sleep of fast caffeine metabolizers.

It is an object of the invention to provide a dietary sleep supplement that mitigates the effects of caffeine prior to sleep.

### SUMMARY

This object is achieved by the composition according to the present invention. A sleep supplement formulation, in accordance with the invention, includes Sulforaphane and one or more of the following: Evodia Fruit Extract, Vitamin C, Quercetin, Red Sage, Angelica Dahruica Root, Golden Seal, L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract.

According to one embodiment, a sleep supplement formulation consists essentially of Sulforaphane and one or more of the following: Evodia Fruit Extract, Vitamin C, Quercetin, Red Sage, Angelica Dahruica Root, Golden Seal, L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract. This formulation may be in any ingestible form, such as a powder, a capsule, a tablet, a gummy, or a liquid, and may be designed to be added to either another dietary supplement or to a food or drink.

According to certain embodiments, the sleep supplement formulation includes between 0.0001 and 0.2 wt % Sulforaphane.

The sleep supplement formulation may be in any ingestible form, such as a powder, a capsule, a tablet, a gummy, or a liquid, and may be designed to be added to either another dietary supplement or to a food or drink.

A method of combatting or mitigating effects of caffeine on a person's sleep includes orally administering to the person a sleep supplement formulation that includes Sulforaphane.

### DETAILED DESCRIPTION

Compositions and methods described herein solve the problem of providing a dietary sleep supplement that mitigates the effects of caffeine prior to sleep. The sleep supplement formulation includes Sulforaphane and one or more of the following: Evodia Fruit Extract, Vitamin C, Quercetin, Red Sage, Angelica Dahruica Root, Golden Seal, L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract. The sleep supplement formulation can be taken a few hours prior to sleep, such as between 1 and 4 hours prior to sleep, to enable the body to speed up the evacuation of caffeine from the body in order to facilitate deep sleep.

Sulforaphane is a sulfur-rich, natural plant compound found in many cruciferous vegetables, including broccoli, cabbage, bok choy, cauliflower, and kale. With its antioxidant, antimicrobial, and anti-inflammatory properties, it has been shown to provide powerful health benefits. Studies have shown that Sulforaphane can significantly reduce caffeine levels in humans.

Vitamin C, also known as ascorbic acid, is a water-soluble vitamin found in citrus fruits as well as other fruits and vegetables. Like Sulforaphane, Vitamin C also provides powerful health benefits. Vitamin C is available in a buffered form, such as Calcium Ascorbate or Magnesium Ascorbate. Other forms of Vitamin C include Ascorbyl Palmitate and Sodium Ascorbate.

Quercetin is a polyphenol found in many fruits, vegetables, leaves, seeds, and grains. Quercetin has antioxidant and anti-inflammatory effects. Quercetin is available in standard form, as Enzymatically Modified Isoquercitrin (EMIQ), as well as in the form of Phytosome® available from Indena S.p.A. in Milan, Italy.

Evodia Fruit Extract is a botanical extract derived from the fruit of the evodia tree (Evodia rutaecarpa). This extract is also known as Tetradium rutaecarpa fruit extract or Wu Zhu Yu extract. Evodia Fruit Extract is often used as a skin conditioning agent in cosmetic formulations. Known for its anti-inflammatory properties, Evodia Fruit Extract is also used in traditional Chinese medicine. Studies have shown that Evodia Fruit Extract can significantly reduce caffeine levels in mice.

According to one embodiment, the sleep supplement formulation includes two sets of ingredients, namely, one set that aids in the elimination of caffeine and one set that aids in inducing sleep and maintaining sleep throughout the night. The caffeine-abating ingredients may include one or more of the following: Sulforaphane, Evodia Fruit Extract, Vitamin C, Quercetin, Red Sage, Angelica Dahruica Root, and Golden Seal. The sleep-assisting ingredients may include one or more of the following: L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract. Additional ingredients, such as hydroxypropyl methylcellulose, microcrystalline cellulose, and/or magnesium stearate, may be added in order to develop the sleep supplement formulation into the desired form.

The amount of Sulforaphane in the formulation may be between 0.05 and 10 mg, or between 0.1 and 5 mg, or between 0.1 and 0.9 mg. The formulation may also include any one or more of the following: 10-2000 mg Vitamin C; 50-500 mg Quercetin; 50-1500 mg Red Sage; 500-6000 mg Angelica Dahruica Root; and 100-1500 mg Golden Seal. The formulation may also include between 25 and 300 mg, or between 50 and 250 mg, or between 50 and 150 mg Evodia Fruit Extract.

With respect to Vitamin C, more particularly, the formulation may include 150-300 mg Calcium Ascorbate, 150-300 mg Magnesium Ascorbate, 50-100 mg Ascorbyl Palmitate, or 150-250 mg Sodium Ascorbate. For example, the formulation may include 200 mg Magnesium Ascorbate.

With respect to Quercetin, the formulation may include 150-250 mg Quercetin, such as in the form of Phytosome® available from Indena S.p.A. in Milan, Italy, or 50-100 mg Enzymatically Modified Isoquercitrin (EMIQ), or 300-500 mg standard Quercetin.

The formulation may include any one or more of the following: 1-200 mg, 2.5-50 mg, or 5-15 mg Vitamin B6, such as in the form of Pyriodoxine HCl; 25-500 mg, 25-250 mg, or 25-75 mg Magnesium, such as in the form of Magnesium Malate; 5-80 mg, 10-50 mg, or 10-30 mg Zinc, such as in the form of Zinc Picolinate; 50-1000 mg, 100-500 mg, or 100-300 mg L-Tryptophan; 50-900 mg, 50-400 mg, or 100-300 mg L-Theanine; 1-10 mg Melatonin; 100-1000 mg Ashwagandha Leaf and Root Extract; 100-1600 mg Chamomile Flower Extract; 10-3000 mg or 50-1000 mg Gamma Aminobutyric Acid (GABA); 200-6000 mg Glycine; 300-600 mg Valerian Root; 300-1600 mg Lemon Balm Extract; for example. According to certain embodiments, the formulation may include between 1 and 2 wt % Vitamin B6, between 5 and 10 wt % Magnesium, between 2 and 5 wt % Zinc, between 30 and 50 wt % L-Tryptophan, between 20 and 40 wt % L-Theanine, between 0.2 and 2 wt % Melatonin, between 15 and 30 wt % Evodia Fruit Extract, and/or between 0.0001 and 0.2 wt % Sulforaphane.

The sleep supplement formulation may be provided in virtually any ingestible form, such as a powder or liquid that can be added to food or drink, or a capsule such as a gel capsule, or a gummy, or a tablet. The amounts of each ingredient listed herein is per serving, which may be per capsule or per tablet or per gummy or per measured amount of powder or liquid, or per two capsules, tablets, or gummies, depending on the size and form of the supplement.

According to another embodiment, the sleep supplement formulation may be formed solely of Sulforaphane and one or more of the following: Evodia Fruit Extract, Vitamin C, Quercetin, Red Sage, Angelica Dahruica Root, Golden Seal, L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract. This formulation may be designed to be added to another dietary supplement. This formulation may also be provided in virtually any ingestible form, such as a powder or liquid that can be added to food or drink, or a capsule such as a gel capsule, or a gummy, or a tablet.

A method of combatting or mitigating the effects of caffeine on a person's sleep includes orally administering to the person the sleep supplement formulation described herein. For example, one or two supplements, which may be around 600-700 mg each, in the form of a gel capsule may be taken by an adult 0 to 12 hours after consuming caffeine.

### EXAMPLE

One example formulation in accordance with the invention is shown below in Table 1:

TABLE 1
Sample Formulation

| | Amount |
|---|---|
| Ingredient | per serving |
| Vitamin B6 (as Pyridoxine HCl) | 10 mg |
| Magnesium (as Magnesium Malate) | 53.2 mg |
| Zinc (as Zinc Picolinate) | 20 mg |
| L-Tryptophan | 250 mg |
| L-Theanine | 200 mg |
| Evodia Fruit Extract | 100 mg |
| Melatonin | 4 mg |
| Sulforaphane (from BroccoPhane ® | 0.4 mg |
| Broccoli Sprout Extract) | |

The composition in Table 1 may include minor amounts of additional ingredients, such as one or more of the following: hydroxypropyl methylcellulose, microcrystalline cellulose, and magnesium stearate. The composition in Table 1 is appropriate for a single daily dose for an adult.

The descriptions included herein depict specific implementations to teach those skilled in the art how to make and use the best option. For the purpose of teaching inventive principles, some conventional aspects have been simplified or omitted. Those skilled in the art will appreciate variations from these implementations that fall within the scope of the invention. Those skilled in the art will also appreciate that the features described above can be combined in various ways to form multiple implementations. As a result, the invention is not limited to the specific implementations described above, but only by the claims and their equivalents.

1. A sleep supplement formulation comprising:
Sulforaphane; and
at least one of the group consisting of Evodia Fruit Extract, Vitamin C, Quercetin, Red Sage, Angelica Dahruica Root, Golden Seal, L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract.

2. The sleep supplement formulation according to claim 1, comprising between 0.05 and 10 mg Sulforaphane per serving.

3. The sleep supplement formulation according to claim 1, comprising Vitamin C.

4. The sleep supplement formulation according to claim 1, comprising Quercetin.

5. The sleep supplement formulation according to claim 1, wherein the formulation is in a form selected from the group consisting of a powder, a capsule, a tablet, a gummy, and a liquid.

6. A method of mitigating effects of caffeine on a person's sleep, comprising:
orally administering to the person the sleep supplement formulation according to claim 1.

7. A sleep supplement formulation comprising:
at least one caffeine-abating ingredient, wherein the at least one caffeine-abating ingredient includes Sulforaphane; and
at least one sleep-assisting ingredient selected from the group consisting of L-Tryptophan, L-Theanine, Melatonin, Vitamin B6, Magnesium, Zinc, Ashwagandha Leaf and Root Extract, Chamomile Flower Extract, Gamma Aminobutyric Acid (GABA), Glycine, Valerian Root, and Lemon Balm Extract.

8. The sleep supplement formulation according to claim 7, wherein the at least one caffeine-abating ingredient further comprises at least one of the group consisting of: Evodia Fruit Extract, Vitamin C, Quercetin, Red Sage, Angelica Dahruica Root, and Golden Seal.

9. The sleep supplement formulation according to claim 7, comprising between 0.05 and 10 mg Sulforaphane per serving.

10. The sleep supplement formulation according to claim 7, wherein the formulation is in a form selected from the group consisting of a powder, a capsule, a tablet, a gummy, and a liquid.
